(19) United States
(12) Patent Application Publication
(10) Pub. No.: US 2013/0040958 A1
(43) Pub. Date: Feb. 14, 2013

(54) IMATINIB MESYLATE PREPARATION PROCEDURE

(71) Applicants: Ennio GRENDELE, Valdagno (IT); Marco GALVAGNI, Verona (IT)

(72) Inventors: Ennio GRENDELE, Valdagno (IT); Marco GALVAGNI, Verona (IT)

(73) Assignee: F.I.S. Fabbrica Italiana Sintetici S.p.A., Alte di Montecchio Maggiore (IT)

(21) Appl. No.: 13/546,058

(22) Filed: Jul. 11, 2012

(30) Foreign Application Priority Data
Jul. 14, 2011 (IT) MI2011A001309

(51) Int. Cl. A61K 31/506; A61P 35/02; B02C 23/18; C07D 401/14

(52) U.S. Cl. 514/252.18; 544/295; 241/21

(57) ABSTRACT

Methods for making Imatinib mesylate of formula (I):

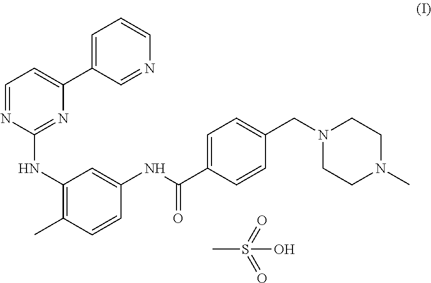

having low levels of organic solvent are provided. Also provided are alpha and beta forms of Imatinib mesylate and methods for treating conditions such as chronic myelogenous leukaemia by administering same.

### CROSS REFERENCE TO RELATED APPLICATION

This application claims priority to and benefit of Italian Patent Application No. MI2011A001309 filed Jul. 14, 2011, the contents of which are incorporated by reference in their entirety.

### FIELD OF THE INVENTION

The present invention relates to methods for the preparation of Imatinib mesylate.

### BACKGROUND OF THE INVENTION

Imatinib mesylate, compound of formula (I), is an important active ingredient used to treat chronic myelogenous leukaemia.

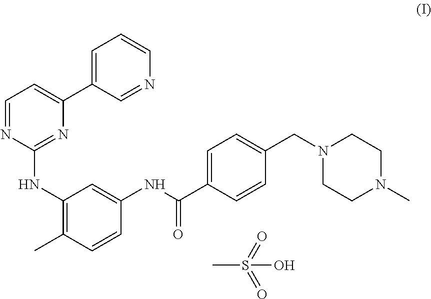

Its preparation was described for the first time in Novartis' EP 564409 and subsequently in EP998473 where the preparation of Imatinib mesylate alpha and beta polymorphic form are described. An interesting and inexpensive procedure for the preparation of the key intermediate is described in WO2008/059551 in the name of F.I.S. Fabbrica Italiana Sintetici S.p.A. Also described are other preparations of polymorphic forms of Imatinib mesylate, in particular the alpha and beta forms, in WO2006/024863, US2006/0223816, WO2008/150481 and WO2009/151899. None of these references discuss the significant problem relating to residual solvents. Such residual solvents do in fact represent impurities of the active ingredient and of the formulated product like typical organic impurities. Such impurities to conform to ICH and related national and European guidelines, must be quantified and comply with the relative limits.

### SUMMARY OF THE INVENTION

The problem addressed by the present invention is therefore that of providing a procedure for the preparation of Imatinib mesylate of formula (I) having a residual solvent content in conformity with current ICH guidelines and regulatory standards throughout the world.

Such problem is solved by methods for the synthesis of Imatinib mesylate as described herein and in the claims.

### BRIEF DESCRIPTION OF THE FIGURES

FIG. 1 illustrates the rotor-stator principle,

FIG. 2 shows an embodiment of the present invention at laboratory scale with an appropriate disperser.

FIG. 3 illustrates a part of an industrial disperser wherein the three in-series rotor-stator combinations can be seen, which facilitate the breaking up into small pieces and the homogenization of the product in suspension.

### DETAILED DESCRIPTION OF THE INVENTION

The present invention relates to methods for the preparation of Imatinib mesylate of formula (I):

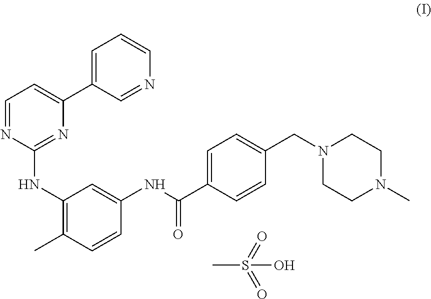

having a residual solvent content in compliance with the guidelines of the ICH (International Conference of Harmonization) Q3C(R4) (Impurities: guideline for Residual Solvents) published in February 2009. A particular reference is made with respect to the solvents classified in Class 3 for which a residual solvent limit of 5000 ppm is required.

Experimentally repeating the preparatory methods described in the current state of the art, though obtaining the product in the alpha and beta polymorphic forms, the problem of the residual solvent content was consistently greater than the ICH limits. The problem was evident even when different types of solvents were used, e.g., alcohols, ketones, acetates, ethers, etc. It was then unexpectedly discovered that by treating a suspension of Imatinib mesylate in an organic solvent with a disperser or crusher comprising one or more stator combinations and one or more rotors, Imatinib mesylate can be obtained having residual solvents in compliance with the ICH guidelines and pharmacopoeias.

By preparing Imatinib mesylate, in both alpha and beta polymorphic form, e.g., crystallizing or re-crystallizing it from n-butanol, a product is obtained containing over 17000 ppm of residual n-Butanol even if the product is dried at 60° C. under vacuum for 48 hours. By drying the product at 120° C. under vacuum for 24 hours or under nitrogen flow at 90° C., the product still contains 9000-10000 ppm of residual n-Butanol which is much higher than the 5000 ppm of the ICH limit. Totally similar problems, with the same levels of residual solvents, can be observed by crystallizing the product, for example, from Ethanol, Isopropanol, MEK or MTBE. Using the procedure of the present invention, employing for example n-butanol, even by drying the product at just 60°-80° C. for 10-12 hours, Imatinib mesylate may be obtained having less than 5000 ppm of n-Butanol, normally around 3000 ppm and in any case always above 1000 ppm and below 5000 ppm, and is therefore in compliance with the current ICH limits for such solvent.

Without being bound by the following theory, a possible scientific explanation of the effect of the present invention has been found by discovering that the product crystallizes by combining solvent molecules inside the crystal aggregates. It has thus also been confirmed by means of DSC and TGA measurements that the solvent does not enter the crystal lattice to form a solvated species. Instead solvent is simply combined in the crystal aggregate. Nevertheless it is very difficult to remove or bring it within permitted limits using conventional drying methods. On an industrial scale, the problem of drying the product in such a way as to lead to a product of pharmaceutically acceptable grade has dragged on for quite a long time without any industrially viable solutions being found.

The procedure of the present invention is therefore achieved by a treatment of a suspension of Imatinib mesylate with a crusher or disperser able to break down the particles of product in suspension. The grinding treatment carried out on the solid dried or partially dried product does not produce the same results inasmuch as the quantities of residual solvents remain high.

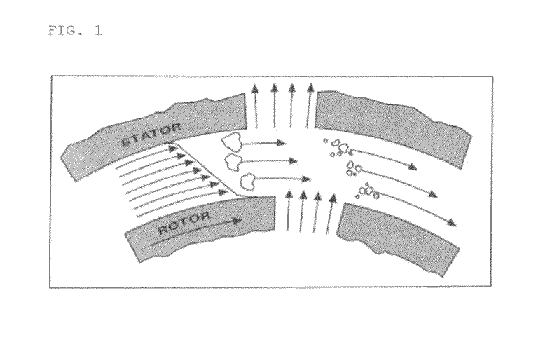

Appliances have been available for a long time at both laboratory level and industrial level which are able to perform this product dispersion procedure by splittin up and distributing the solid in the liquid phase, thereby obtaining a homogeneous suspension. The principle on which these appliances are based is called rotor-stator principle and is exemplified in FIG. 1 and summed up as follows. The high number of rpm of the rotor axially sucks up the fluid into the dispersion head and is then pushed radially through the slots of the rotor-stator. The strong accelerating forces apply very strong tension and thrust forces on the material. In addition, in the slot between the rotor and the stator, a strong turbulence is produced that causes the suspension to mix in the best possible way. These types of appliances can be called dispersers, crushers, homogenizers or, simply, mixers.

Methods of the present invention may utilize a crusher/disperser which comprises one or more stator combinations and one or more rotors and operates between about 1000 and about 100,000 rpm.

For laboratory applications, the best tip speed of the rotor-stator system is around 6-30 m/s, preferably about 23 m/s and rotor ranges of between about 3000 and about 30,000 rpm. Imatinib mesylate therefore may be subjected to such tip speed and rotor rpm values. In the case of industrial applications, speeds of up to 100,000 rpm can even be reached, even though, especially for very large scales, rotor speeds of 1000-2000 rpm are normally applied.

For laboratory scale applications, 8000-10000 rpm give satisfactory results. Preferably, ranges between 1000 and 10000 rpm are therefore generally used.

For laboratory applications in general, a treatment time of just a few minutes is enough to achieve the required end fineness. Longer treatment times only provide marginal improvement to the fineness of the materials and consequently 15-minute treatments are normally enough.

A specific type of laboratory equipment suitable for methods according to the present invention is the IKA ULTRA-TURRAX T25 digital.

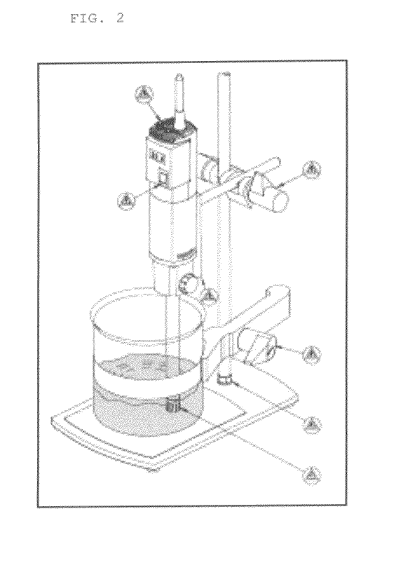

In this appliance (FIG. 2) there is just one rotor-stator combination.

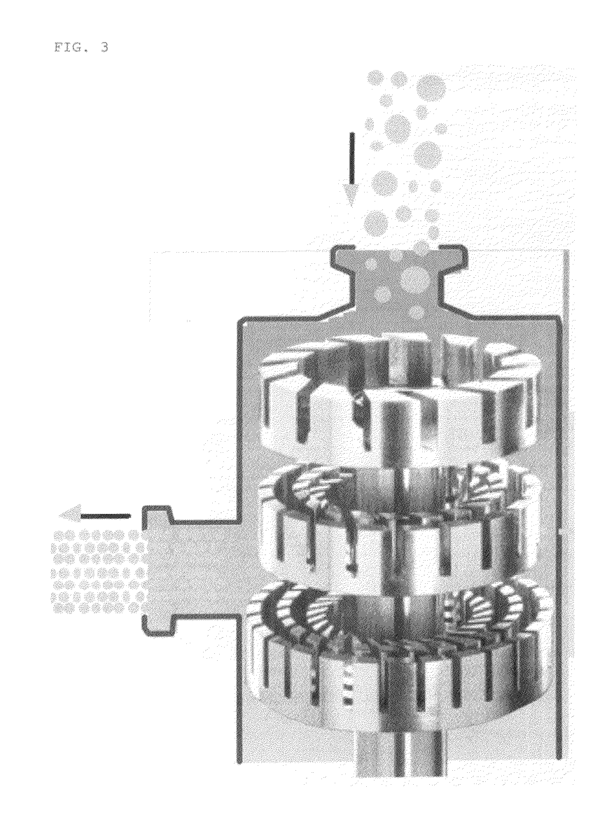

For industrial applications, the IKA® Dispax Reactor model DR 2000/10 Disperser is particularly effective inasmuch as it has a 3-stage rotor-stator system consisting of 3 rotor-stator combinations in series. (See FIG. 3.) Such industrial appliances also take the name of “wet mill”.

For laboratory-scale preparations, the procedure may be performed with treatment times between about 5 and about 60 minutes. In certain embodiments, the treatment time may be about 15 minutes.

The organic solvent in which the Imatinib mesylate can be conveniently suspended or may be obtained may be chosen from among alcohols, ketones, acetates and ethers. For example, the Imatinib mesylate may be suspended in a C1-C5 alcohol and therefore in an alcohol chosen from among methanol, ethanol, n-propanol, isopropanol, n-butanol, sec-butanol, isobutanol, tert-butanol, n-pentanol, 2-ethy-1-1-propanol, 2-methy-1-1-butanol, 3-methy-1-1-butanol, isopentanol, sec-pentanol, 2,2-dimethy-1-1-propanol. In certain embodiments, methods of the present invention may be conducted by preparing a suspension of Imatinib mesylate in ethanol or isopropanol or n-butanol.

In certain embodiments, the methods of the invention may be conducted by preparing a suspension of Imatinib mesylate in n-butanol. This solvent has the advantage of allowing slow crystallization, for example, slower than isopropanol and the other lower alcohols, wherein the crystallization of the product is very fast. N-butanol, therefore, permits, depending on the crystallization conditions, obtaining both the alpha or the beta form of Imatinib mesylate.

The procedure of the present invention thus allows for the preparation of Imatinib mesylate of formula (I):

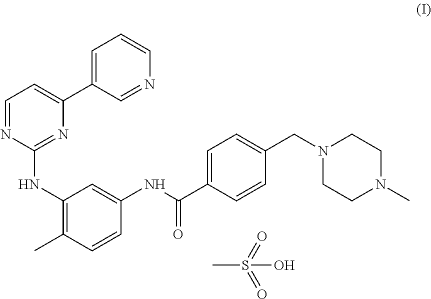

having between about 1000 and about 5000 ppm of ethanol, propanol, butanol or pentanol. In particular, propanol may be selected from n-propanol and isopropanol; butanol may be selected from n-butanol, isobutanol, sec-butanol, tert-butanol; and pentanol may be selected from n-pentanol, isopentanol and sec-pentanol.

In particular, by using n-butanol it is possible to obtain Imatinib mesylate having a residual content of solvent between about 1000 and about 5000 ppm and thus in conformity with the present ICH limits and in certain embodiments between about 2000 and about 4000 ppm.

Furthermore, the Imatinib mesylate may be obtained in both alpha and beta polymorphic form (as characterized in EP998473).

The procedure of the present invention may be applied both during product crystallization and during a subsequent product re-crystallization or crushing stage. The content of residual solvents is an essential characteristic for pharmaceutical products, whether these are active ingredients or formulated products, inasmuch as compliance with appropriate limits for pharmaceutical products is obligatory.

Moreover, Imatinib mesylate obtained according to embodiments of the present invention by using n-butanol as a crystallization or recrystallization solvent is free of any detectable amount of the genotoxic impurity n-butylmesylate. (The detectability threshold is 0.1 ppm.)

Imatinib mesylate prepared according to methods of the present invention can therefore be conveniently formulated with one or more acceptable pharmaceutical excipients. The formulated product may be used in medicine and, specifically, can be used to cure and/or treat chronic myelogenous leukaemia.

### EXAMPLES

### Example 1

### Synthesis of Imatinib mesylate (I) Alpha Form

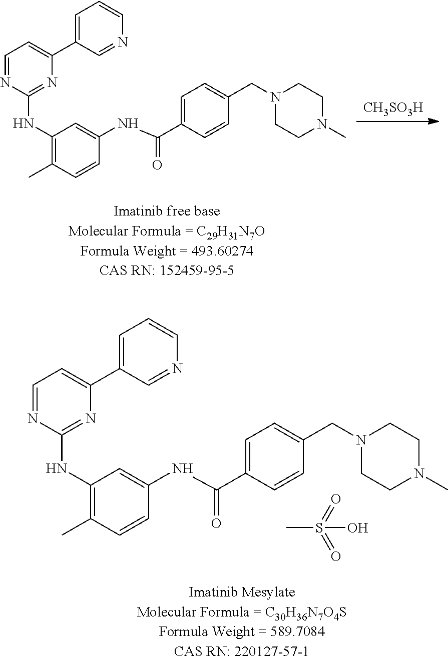

In a 20-litre steel vessel 400.0 g of Imatinib base (prepared according to example 21 of EP0564409) and 8000 ml of n-butanol were loaded under nitrogen flow and heated under reflux. 20.0 g of Carbon eno-pc were added and the contents were shaken at reflux temperature for 15 minutes, before filtering on steryflon ptf2071 sl cartridge and conveying the filtrate by nitrogen thrust to a preheated glass reactor. A yellowish solution was obtained at pH=6.4. Afterwards, 75.8 g (51.20 ml) of methanesolfonic acid were dripped in one minute at T=80° C. A clear solution was obtained at approx. stable 4.5 pH. This was shaken and left to cool at T=70° C. and primed with 5.0 g of Imatinib mesylate, alpha form.

Precipitation with rapid thickening was observed. The solution was shaken for one hour and left to cool at T=20° C. This was filtered in 5 minutes and washed with 680.0 ml of n-Butanol. It was then dried in a vacuum stove at T=50° C. until constant weight, and 315 g of Imatinib mesylate, alpha form were obtained with a molar yield of 65.7%. The product contained over 17,000 ppm of residual n-Butanol determined by GC-HS.

### Example 2

### Synthesis of Imatinib mesylate (I) Alpha Form

In a reactor under nitrogen 200.0 g of Imatinib mesylate, obtained according to Example 1, and 7000 ml of n-Butanol were loaded and this was heated at reflux before being filtered hot on a dicalite cake. The filtrate was left to cool to 80° C. at which time 20.0 g of Imatinib mesylate, alpha form, primer were added and treated for 15 minutes with a high-performance IKA disperser at around 7500 rpm (as shown in FIG. 2). This was shaken and left to cool at T=40° C. for 30-40 minutes before being filtered and washed with a little n-butanol. The solid was then dried at 70-80° C. for 10-12 hours. 174.0 g of product were obtained, equivalent to a molar yield of 79.1%. The content of residual n-butanol was around 3000 ppm.

### Example 3

### Synthesis of Imatinib mesylate (I) Beta Form

The same procedure was followed as described in Example 1 except that the primer was made with 5.0 g of Imatinib mesylate beta form instead of alpha form.

About 318 g of Imatinib mesylate beta form were obtained for a molar yield of 66.3%.

### Example 4

### Synthesis of Imatinib Mesylate (I) Beta Form

The same procedure was followed as described in Example 2 except that the primer was made with 20.0 g of Imatinib mesylate beta form instead of alpha form.

190.3 g of Imatinib Mesylate Beta form were obtained with a molar yield of 86.5%.

The content of residual n-butanol was around 3000 ppm.

### Example 5

### GC-HS Method for Residual Solvent Determination in Imatinib Mesylate

### Chromatographic Conditions:

Column^{.} Fused silica , L =30 m, ID =0.54 mm, FT=3 μm
Liquid phase: DB-624
InjectorTemp: 200° C.
Detector Temp: 250° C.
ColumnTemp: 40° C. for 5 min., from 40° C. to 90° C. at 4° C./min. from 90° C. to 230° C. at 20° C./min, isotherm at 230° C. for 6 min.
Injection: split, split ratio 1:1 *
Carrier gas: Helium, constant flow (flow=4.5 mL/min)*
Detector: Flame ionization (air 400 mL/min.; Hydrogen 40 mL/min *
Make up: Nitrogen (flow 30 mL/min.)
Diluent: Dimethylsulphoxide
Vialsize: 20 mL
Sample Volume: 5 mL
   *These parameters may be modified to optimize the signal or to reach the requested performance

### Head-Space Conditions:

| | | |
|---|---|---|
| Oven Temperature: | 100° | C. |
| Loop Temperature: | 200° | C. |
| Transfer Line Temperature: | 200° | C. |
| Vial Equilibration Time: | 20 | min |
| Pressurization Time: | 0.2 | min |
| Vial Pressurization: | 6 | psi |
| Loop Filling Time: | 0.2 | min |
| Loop Equilibration Time: | 0.1 | min |
| Injection Time: | 1 | min |
| ShakingSpeed: | | Low |
| GC cycle time | 40 | min |

### Example 6

### Comparative Examples

The following experimental data were obtained. Further to the comparison between the first two results (the starting product is the same for all the subsequent experimental tests), the data show the difficulties addressed by the present invention.

| | n-butanol |
|---|---|
| Test | content |
| Starting product used for all the tests described below - | 21184 |
| Imatinib masylate beta form | |
| Milling on a mortar + drying in stove T = 60° C. for 4 days | 17900 |
| Pulping at RT in n-Butanol + drying in stove T = 60° C. for | 17600 |
| 4 days | |
| Pulping at RT in n-Eptane + drying in stove under vacuum | 19000 |
| T = 90° C. for 30 h | |
| Recrystallization from EtOH + H2O | 11600 |
| Pulping in AcOEt | 22700 |
| Treatment for 24 hr under high vacuum | 17950 |
| Treatment for 24 hr at 120° C. | 19000 |
| Pulping at reflux in n-Eptane + drying in stove T = 60° C. | 17800 |
| Recrystallization from n-Butanol + drying at T = 120° C. | 7890 |
| under vacuum for 15 hr. | |

Similar results were obtained starting from Imatinib masylate alpha form.

It is thus evident that, even if it is known to a person skilled in the art that reduction in the amount of residual solvents in a product can be achieved by milling to reduce the particle size, this process is ineffective with respect to Imatinib masylate. In contrast with most active agents, Imatinib masylate has the unusual tendency to retain the residual solvents, particularly alcoholic solvents. Milling does not provide for an effective reduction of their amount.

In particular, it should be appreciated that the use of the conditions forming the subject of the present invention permit obtaining Imatinib mesylate of formula (I) both in alpha and beta polymorphic form having a residual solvent content in compliance with ICH guidelines.

1. A method for the preparation of Imatinib mesylate of formula (I): 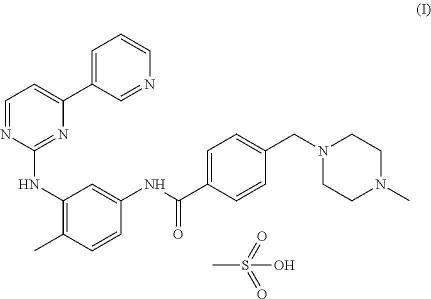
comprising introducing a suspension of Imatinib mesylate in an organic solvent into a crusher/disperser comprising one or more stator combinations and one or more rotors.

2. The method of claim 1, wherein one or more rotors are set at a speed between about 1000 and about 100,000 rpm.

3. The method of claim 1, wherein the Imatinib mesylate is subjected to a tip speed of around 23 m/s.

4. The method of claim 1, wherein said method is performed between about 5 and about 60 minutes.

5. The method of claim 4, wherein said method is performed for about 15 minutes.

6. The method of claim 1, wherein the organic solvent is selected from the group consisting of: alcohols, ketones, acetates and ethers.

7. The method of claim 6, wherein the organic solvent is a C1-C5 alcohol.

8. The method of claim 7, wherein the organic solvent is selected from the group consisting of: ethanol, isopropanol, and n-butanol.

9. A Imatinib mesylate compound of formula (I): 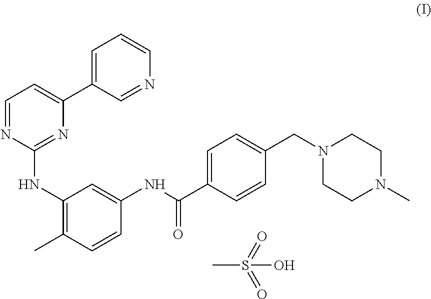
comprising between about 1000 and about 5000 ppm of an organic solvent selected from the group consisting of: ethanol, propanol, butanol and pentanol.

10. The Imatinib mesylate of claim 9, wherein the propanol is selected from the group consisting of: n-propanol and isopropanol; the butanol is selected from the group consisting of: n-butanol, isobutanol, sec-Butanol, tert-Butanol; and the pentanol is selected from the group consisting of: n-pentanol, isopentanol and sec-pentanol.

11. The Imatinib mesylate of claim 9, comprising n-butanol.

12. The Imatinib mesylate of claim 11, comprising between about 2000 and about 4000 ppm of n-butanol.

13. The Imatinib mesylate of claim 9, wherein the Imatinib mesylate is in the alpha polymorphic form or the beta polymorphic form.

14. A pharmaceutical composition comprising the Imatinib mesylate of claim 9 and one or more acceptable pharmaceutical excipients.

15. A method for treating chronic myelogenous leukaemia comprising the administration of the compound of claim 9 to a subject in need of such treatment.

16. A method for preparing Imatinib mesylate having less than 5000 ppm of a residual organic solvent comprising use of a crusher/disperser having one or more stator combinations and one or more rotors.
